(19) United States
(12) Patent Application Publication
(10) Pub. No.: US 2006/0110400 A1
(43) Pub. Date: May 25, 2006

(54) Immunogenic conjugates

(75) Inventors: James Glover, Runcorn (GB); Matthew Downham, Runcorn (GB); Ajaz Rosul, Runcorn (GB); Deborah Spencer, Runcorn (GB); Iain Hargrave, Runcorn (GB)

Correspondence Address: BACON & THOMAS, PLLC, 625 SLATERS LANE, FOURTH FLOOR, ALEXANDRIA, VA, 22314, US

(73) Assignee: PROTHERICS PLC, Runcom (GB)

(21) Appl. No.: 10/517,306

(22) PCT Filed: Jun. 17, 2003
(86) PCT No.: PCT/GB03/02596

§ 371 Date: Dec. 20, 2005

(30) Foreign Application Priority Data
Jun. 17, 2002 (GB) 0213878.2

(51) Int. Cl. A61K 39/00; C07H 21/04; C12P 21/04; C07K 14/475

(52) U.S. Cl. 424/185.1; 530/399; 435/69.7; 435/320.1; 435/325; 536/23.5

(57) ABSTRACT

The invention relates to an immunogenic conjugate comprising at least one vascular endothelial growth factor (VEGF) peptide moiety coupled to a carrier.

The present invention relates to immunogenic conjugates of mammalian vascular endothelial growth factor (VEGF) or fragments, analogs or derivatives thereof, and to their use for the therapy and prophylaxis of conditions associated with tumour angiogenesis.

VEGF is a potent mitogen which induces vasculogenesis and angiogenesis. Vasculogenesis is the de novo development of blood vessels through differentiation of early endothelial cells during embryogenesis. Angiogenesis on the other hand is the formation of new blood capillaries from pre-existing blood vessels through new tissue. VEGF is therefore found in damaged tissues where VEGF induction by hypoxia subsequently causes angiogenesis as part of the repair mechanism. VEGF is also involved in the monthly cyclical regeneration of the endometrium in pre-menopausal women.

Native VEGF demonstrates considerable sequence homology across a range of species, including humans and rodents. Typically it is a glycoprotein produced by dimerisation of alternative spliced variants of a single gene. Polypeptide chains of 121, 165, 189 and 206 amino acid residues have been identified (Houck, K. A. et al 1996; Mol. Endocrinol. 5: 1806-1814). Of these, the 121 amino acid residue isoform VEGF₁₂₁ has been demonstrated to be the most potent molecule in angiogenesis.

We have now found that derivatives of VEGF can be produced which are potent immunogens and which can be used in an immunotherapeutic approach to combat conditions associated with tumour angiogensis. In particular, derivatives of VEGF have been developed in which one or more VEGF peptide moieties are coupled to a carrier, such as a protein or polypeptide to form an immunogenic conjugate. The conjugate immunogen is capable in an immunised host of inducing antibodies which bind to native VEGF and neutralise its effects.

Thus viewed from one aspect the invention provides the use of an immunogenic conjugate comprising at least one vascular endothelial growth factor peptide moiety coupled to a carrier for the manufacture of a medicament for use in combatting tumours, in particular tumour metastasis, e.g. for use in combatting tumour angiogensis through vascularisation and thus controlling the spread of cancer by limiting metastasis.

Viewed from a further aspect the invention provides an immunogenic conjugate comprising at least one vascular endothelial growth factor peptide moiety coupled to a carrier, e.g. for use in therapy or prophylaxis, particularly to combat tumours and tumour metastasis.

The VEGF conjugates may be used to immunise a patient against the native VEGF molecule such that the activity of the growth factor is neutralised by specific anti-growth factor or antipolypeptide antibodies.

The VEGF peptide moiety may be any peptide moiety, without necessarily having the biological activity of a native VEGF in the body, which is capable of acting as an immunomimic of native VEGF, i.e. which immunologically mimics VEGF so as to generate antibodies which bind to and incapacitate the native VEGF molecule.

Such peptide moieties may include modifications of the native VEGF sequence by single or multiple amino acid substitution, addition or deletion and also sequences where the amino acid residues are chemically modified, but which nonetheless retain VEGF immunogenic activity. Such functionally (i.e. immunologically) equivalent variants may occur as natural biological variations, or they may be prepared using known and standard techniques for example by chemical synthesis or modification, mutagenesis e.g. site-directed or random mutagenesis etc. The important feature as regards the modification is that the VEGF peptide retains the ability to act as immunomimic of native VEGF. Thus for example, an amino acid may be replaced by another which preserves the physicochemical character of the VEGF peptide or its epitope(s) e.g. in terms of charge density, hydrophilicity or hydrophobicity, size and configuration and hence preserve the immunological structure. “Addition” variants may include N- or C-terminal fusions as well as intrasequence insertion of single or multiple amino acids. Deletions may be intrasequence or may be truncation from the N- or C-termini. The term “VEGF peptide” as used herein includes all native VEGF peptides and their functionally equivalent variants.

In general the VEGF peptide moiety will comprise an amino-acid sequence having a high degree of homology, e.g. at least 80% homology, preferably at least 90% homology, with the whole or a section of a native VEGF sequence, e.g. section of 8 to 100, more preferably 10 to 30, especially preferably 12 to 25 amino acids. Particularly preferably it has this high degree of homology with a section of a native VEGF sequence overlapping, abutting or adjacent (e.g. within 5 amino acid residues of) a glycosylation site of the native VEGF sequence, especially a section which includes at least 8 of the first 12 amino acid residues from such a glycosylation site in the N-terminal direction, more especially at least 12 of the first 16 amino acid residues from such a glycosylation site in the N-terminal direction.

Most preferably, the VEGF peptide moiety comprises an oligopeptide with a high degree of homology (preferably 100% homology) with at least part of the 71-100 section of VEGF₁₂₁ shown below as SEQ ID No.1

SEQ ID No.1

TEESNITMQI MRIKPHQGQH IGEMSFLQHN

The VEGF peptide moiety preferably comprises an oligopeptide (T)ₐ(M)_{b}(O)_{c}(I)_{d} MRIKPHQGQ(H)ₑ(I)_{f}(G)_{g}(E)ₕ(M)ᵢ(S)ⱼ(F)ₖ(L)ₗ(O)ₘ where a to m are each 0 or 1, but a to c and f to m may not be 1 unless the sequence so created corresponds to, a sequence in SEQ ID No.1. e to g are preferably 1, more preferably e to j are 1.

The VEGF peptide moiety is preferably coupled via its N-terminal end to the carrier. Where it is coupled via its C-terminal end it is preferably an at least 14-mer, especially one including the sequence HIGEM.

The VEGF peptide moiety is conveniently coupled to the carrier via a carrier-binding moiety. As a precursor to the conjugate one may thus prepare a VEGF derivative comprising at least one VEGF peptide moiety coupled to a peptide carrier-binding moiety. Such derivatives form a further aspect of the invention.

Viewed from a further aspect the invention thus provides a vascular endothelial growth factor derivative comprising at least one VEGF peptide moiety coupled to a peptide carrier-binding moiety.

The carrier-binding moiety serves as a means by which the VEGF peptide moiety may be attached to an immunological carrier, which will generally be a protein or polypeptide, and thus preferably contains an amino acid residue having a reactive side chain, via which the VEGF peptide moiety may readily be coupled to the carrier using standard coupling techniques. Advantageously such a side chain may contain a free hydroxyl, carboxyl or thiol group. Such an amino acid may thus conveniently be a cysteine, tyrosine, aspartic acid or glutamic acid residue or a derivative thereof such as N-acetyl cysteine.

The VEGF derivatives of the invention have been shown to have improved coupling to an immunological carrier for inducing antibodies which can be used immunotherapeutically and these derivatives have advantages in this regard over the native peptide.

The carrier-binding moiety may take the form of a peptide extension at the N- or C-terminal of a VEGF peptide moiety, or a peptide pendant from or disposed within a chain segment between two or more VEGF peptide moieties.

Thus the VEGF derivative may conveniently be of Formula I
((A)-Xₙ)ₘ-Lₚ-Y-[L_{q}(Xᵣ-(A))ₛ]ₜ (I)
Wherein
A represents a VEGF peptide moiety;
X represents an amino acid;
Y represents an amino acid having a side chain with a free —SH, —OH or —COOH group;
L represents an organic linker capable of binding a group (A)-Xₙ)— at one or more sites, e.g. capable of binding up to 10 (A)Xₙ moieties;
n and r are each=0-20;
m and s are each 1, e.g. 1 to 10, preferably 1, 2, 3 or 4; and p, q and t are each 0 or 1; with the proviso that if m 2, then p=1, or if s 2, then q=1.

Preferably A is a 12- to 25-mer VEGF peptide moiety corresponding to a section of SEQ ID No.1. X may be attached at the N- or C-terminus of the VEGF peptide moiety, preferably the N-terminus.

Group L may be any organic linker structure, preferably however, it is a peptide chain, which may be linear or branched or a single amino acid residue, containing residues of natural or synthetic amino acids or pseudo-amino acids. However it may also represent a carboxyl- or amine-terminating dendritic or cascade polymer, for example a branched polyamine.

When t=0, it will be seen that the compounds of Formula (I) include derivatives wherein a carrier binding moiety (i.e. X-Y or X-L-Y) is attached at the N- or C-terminus of a VEGF peptide moiety, as a simple N- or C-terminal extension, or wherein multiple VEGF peptide moieties are linked to a carrier-binding moiety terminating in a group Y, for example as a dendritic array or where the VEGF peptide moieties are attached at multiple sites on the carrier-binding moiety.

When t=1, it will, be seen that the derivatives may take the form of a “dimer”-type structure wherein the carrier-binding group Y of the carrier-binding moiety is disposed within a chain segment of the derivative i.e. effectively between two or more VEGF peptide moieties.

If t=1, and L is an amino acid residue or a peptide chain, L may be or include a “chain-inverting” amino acid or pseudo amino acid (i.e. a compound capable of linking two peptide moieties, e.g. a diamine or dicarboxylic acid), this being a compound capable of inverting or reversing the N- to C-terminal direction of the peptide chain. Such a compound will thus generally include two amino or two carboxylic acid groups, e.g. glutamic acid or a, -alkylene diamine or, -alkylene dicarboxylic acid. When t=1, it is furthermore preferred that the total number of groups ((A)-Xₙ)— does not exceed 8.

Preferred compounds of Formula (I) include those wherein n and r are each 0-10, preferably 1-6, and those wherein m and s are each 8, preferably 1, 2 or 4.

Group X preferably represents an amino acid having no side chain or a hydrocarbyl side chain (preferably an alkyl, C₃₋₇ cycloalkyl or cycloalkenyl, C₃₋₇ cycloalkyl- or cycloalkenyl-alkyl, alkaryl, aralkyl or alkarylalkyl moiety in which each alkyl moiety may be saturated or unsaturated and contains up to 6 carbons and each aryl moiety is preferably a phenyl ring), particularly preferably an aliphatic side chain. Glycine, alanine, -alanine, valine, leucine and isoleucine are preferred and glycine is especially preferred.

Group Y is preferably cysteine, tyrosine, glutamic acid or aspartic acid or a derivative thereof such as N-acetyl-cysteine.

Group L preferably contains at least one residue of an amino acid or pseudo amino acid containing at least two amine or carboxyl groups e.g. lysine, arginine, glutamic acid or aspartic acid, particularly where t=0. Conveniently, such a preferred group L is a linear or branched peptide chain, e.g. containing 2 to 15 amino acid residues. Branching may, of course, occur by peptide bond formation at an amine or carboxyl group of an amino acid residue side chain, e.g. at a side chain amine group of lysine or arginine or at a side chain carboxyl group of aspartic or glutamic acid. A group L comprising one or more, e.g. 1 to 3, lysine residues is especially preferred. Branching may occur by peptide bond formation at both the -amino and -amino groups of lysine.

Preferred compounds of Formula (I) thus include compounds of Formulae (II) to (IV):
(A)-Xₙ-Y (II)
(A)-Xₙ-L-Y (III)
((A)-Xₙ)ₘ-L-Y (IV)
(A)-Xₙ-L-Y-L-Xᵣ-(A) (V)
Wherein A, X, L, n and r are as herein before defined and m=2.

Where the compounds of Formula (IV) contain more than one (A) group, these are preferably attached at the same terminus, i.e. preferably all are N-terminally or all are C-terminally attached. In compounds of Formulae (II) and (III) where X is C-terminally attached to a group A being a VEGF peptide, Y is preferably cysteine. Where X is attached to the N-terminus of A, Y is preferably N-acetyl-cysteine. In Formulae (II) to (V), Xₙ or Xᵣ are each preferably chains of 1 to 6 glycine residues. In compounds of formula (IV), m is preferably 2 or 4. In Formulae (III) to (IV), L is preferably lysine, -lys-(X)ᵤ,-lys-lys-(X)ᵤ, or -lys-lys-(X)ᵤ-lys. Wherein u is 0 to 10, preferably 0 to 6, and X is an amino acid as defined above. Thus, preferred compounds of Formula (IV) are those of Formulae (VI) and (VII): 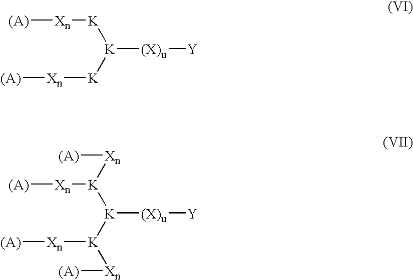
Wherein A, X, Y, n and u are as herein before defined, and K is lysine.

In the “dimer-type” derivatives of Formula (V) the VEGF peptide moiety may be a “reversed” or “inverted” sequence variant of a VEGF peptide ie. a VEGF peptide in which the order of the constituent amino acids is reversed.

Representative VEGF derivatives according to the invention include:
A-(Gly)₁₋₆-Cys;
N-Acetyl-Cys-(Gly)₁₋₆-A; 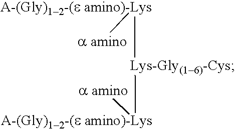
   (The A-(Gly)₁₋₂-moiety may be bonded to either the -amino or the -amino group) 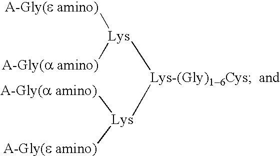
A′-(Gly)₁₋₆-Cys-(Gly)₁₋₆-A;
N-acetyl-Cys-Ala-A
N-acetyl-Cys-(Ala)₄-A
N-acetyl-Cys (Gly)₆-A
N-acetyl-Cys-Gly-Ala-Gly-Ala-A 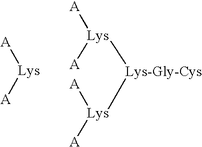
(A)-Gly Cys
(A)-Cys
(A)-Tyr
N-acetyl-Cys-(A)
Tyr-(A)
N-acetyl-Cys-Gly-(A)
Cys-(A)
   Wherein A is VEGF and A′ is VEGF an inverted or reverse VEGF sequence.

Although Glycine is preferred, aliphatic side chain amino acids may be used in place of one or more of the Gly residues in the above formulae.

Although the peptide derivatives of the invention when examined by computer-aided energy minimisation modelling are generally considered too small to be optimally immunogenic alone, it has been found that when coupled via the carrier-binding moiety to a carrier, these peptide derivatives elicit a strong and potentially protective immune response. Their conjugates are thus eminently suitable for use in immunotherapy against tumour angiogenesis. Without wishing to be bound by theory, it is believed that coupling of the peptides to a carrier by means of the carrier-binding moiety results in the derivatives having substantially the same conformation as that of the native VEGF sequences.

The new derivatives according to the invention may be generated using a number of standard techniques including, for peptides, the Merrifield solid phase method in which amino acids are added stepwise to a growing polypeptide linked to a solid matrix (Merrifield, R. B 1962; Fed. Proc. Amer. Soc. Biol. 21: 412 and Merrifield, R. B. 1963; Jour. Amer. Chem. Soc. 85: 2149) and conventional FMOC chemistry. If desired, reactive side chain groups of the amino acids in the growing chain may be protected during the chain synthesis. Branched structures may be prepared by similar techniques.

Where the new derivatives are linear peptides these may also be prepared by recombinant DNA expression using techniques known in the art e.g. as described, for example, by Sambrook et al., in Molecular Cloning: A Laboratory Manual, Second Edition, 1989.

Thus the present invention also provides a nucleic acid molecule coding for the VEGF peptide derivatives of the invention, and nucleic acid molecules with sequences complementary thereto.

According to a further aspect of the invention, we provide an expression vector comprising the said nucleic acid molecule of the invention. Such a vector may be suitable for expression in microorganisms which may be prokaryotic or eurkaryotic e.g. E coli or yeast, or in plant or animal e.g. mammalian cells.

Coupling of the derivative of the invention to the carrier may be by methods known in the art for example by treatment with heterobifunctional linking agents. Where coupling is via a terminal cysteine (or N-acetyl cysteine), the linking agent may be m-Maleimidobenzoyl-N-hydroxysulphosuccinamide ester; in which case maleimide modifies one or more lysine side chains in the peptide carrier, and a thioether bond forms at the terminal cysteine residue. Other coupling reagents known in the art, e.g. carbodiimide coupling, may also be used.

Any carrier known in the art for such purposes may be used, including the purified protein derivative of tuberculin, tetanus toxoid, diphtheria toxoid, keyhole limpet haemocyanin, glutathione S-transferase or derivatives thereof.

Where the VEGF derivative is a linear peptide and the carrier is a protein or polypeptide, the entire peptide conjugate may also be made by recombinant DNA methods wherein a nucleic acid molecule encoding the conjugated molecule is expressed in an appropriate host cell.

The new VEGF conjugates of the invention may be used in an immunotherapeutic approach to combatting diseases associated with normal or elevated levels of angiogenic activity and/or VEGF, and this represents an advantageous method compared to currently available methods. Patient compliance should be increased in that less frequent dosing than is the case with current therapies is involved, and undesirable side effects are avoided.

Thus according to a further aspect, the present invention provides a pharmaceutical composition comprising a VEGF conjugate according to the invention together with one or more pharmaceutically acceptable carriers or excipients.

Viewed from a further aspect, the invention provides a VEGF conjugate according to the invention for use in therapy or prophylaxis.

Viewed from a still yet further aspect, the invention provides a method of comabtting tumours in a human or non-human (e.g. mammalian) subject, comprising administering to said subject an effective amount of a VEGF conjugate according to the invention.

The method may thus be used to control vascularisation of established tumours and prevent the spread of cancer by metastasis.

In one embodiment the method of the invention is a method of immunization against metastasis, i.e. metastases need not already have been detected in the subject. In this embodiment the conjugate may be administered when a tumour is detected, e.g. before it has been determined to be malignant and perhaps before treatment (e.g. chemotherapy, surgery or radiation treatment) of the detected tumour has begun or has been completed.

The VEGF conjugate may be administered by any conventional methods including parentally (e.g. intraperitoneally, subcutaneously, intramuscularly, intradermally for example in the form of inert particles such as gold pellets or beads to which the derivative is adsorbed which may be accelerated at speeds sufficient to enable them to penetrate the skin of a subject, or intravenously), topically (e.g. as a cream to the skin), intra-articularly, mucosally (e.g. orally, nasally, vaginally, rectally and via the intra-ocular route) or by intrapulmonary delivery for example by means of devices designed to deliver the agents directly into the lungs and bronchial system such as inhaling devices and nebulisers, and formulated according to conventional methods of pharmacy optionally with one or more pharmaceutically acceptable carriers or excipients, such as for example those described in Remingtons Pharmaceutical Sciences, ed. Gennaro, Mack Publishing Company, Pennsylvania, USA (1990).

Such compositions are conveniently formulated in unit dosage form e.g. for mucosal, parenteral or oral administration.

Actual treatment regimes or prophylactic regimes, formulations and dosages will depend to a large extent upon the individual patient and may be devised by the medical practitioner based on the individual circumstances.

The type of formulation will be appropriate to the route of administration. For example, parenteral administration by subcutaneous or intramuscular injection may be with a sterile aqueous suspension of the conjugated analogue in PBS, saline or water for injection, optionally together with one or more immunological adjuvants e.g. aluminium hydroxide, saponin, quil A, muramyl dipeptide, mineral or vegetable oils, vesicle-based adjuvants, non-ionic block co-polymers, or DEAE dextran. Additional components such as preservatives may be used.

The dosage for injection may be in the range 1-100 μg peptide equivalent and the frequency of administration may be upwards of from once every three or six months, to once every year or once every five years.

For oral administration, the conjugated derivatives may be formulated as tablets, liquid, capsules etc. Dosages range from 1 to 1000 μg peptide equivalent with dosing occurring at intervals dependent on bio-availability of product.

According to a still yet further aspect, the present invention provides a method for achieving maximal blockade of VEGF in a human or non-human subject comparable to or exceeding that achieved by chemo- or radiotherapy, said method comprising administering to said subject an effective amount of a VEGF conjugate according to the invention.

The VEGF conjugates of the invention moreover may be administered to female humans or non-human mammals as a form of contraception or in the treatment of endometriosis. Such uses, methods etc. form further aspects of the present invention.

The invention will now be described in further detail in the following non-limiting Examples, with reference to the experimental data in which:

Table 2: shows antibody titres +/−sem (dilution corresponding to 0.1 increase in OD) for each of the sera sample days;

Murine model control group.

Group A=Vaccinated with Alhydrogel/Saline placebo.

Group B=Vaccinated with VEGF 6 vaccine.

Group C=Vaccinated with VEGF 7 vaccine.

Group D=Vaccinated with VEGF 8 vaccine.

Group E=Vaccinated with VEGF 9 vaccine.

Table 3: shows effect of each VEGF vaccine on the murine model lung weight following administration of melanoma cells.

Table 4: shows effect of each VEGF vaccine on the development of melanoma colonies in lungs of the murine model.

Tables 2, 3 and 4 have been generated using immunogens prepared from the following derivatives:
N-Acetyl-VEGF 6-Gly-Cys
N-Acetyl-Cys-Gly-VEGF 7
N-Acetyl-VEGF 8-Gly-Cys
N-Acetyl-Cys-Gly-VEGF 9

VEGF 6, 7, 8 and 9 have the sequences of SEQ ID Nos. 2 to 5 respectively.

SEQ ID No.2

TMQIMRIKPHQGQHIGEMS

SEQ ID No.3

TMQIMRIKPHQGQ

SEQ ID No.4

TMQIMRIKPHQGQ

SEQ ID No.5

MRIKPHQGQHIGEMS

### EXAMPLE 1

### Peptide Generation

Peptides were synthesised by the Fmoc strategy of solid phase peptide synthesis on a Protein Technologies, Symphony Peptide Synthesiser. The resin used was Tentagel S-NH2 with a Rink Amide linker. The side chain protecting groups of the Fmoc amino acids used were Trt for Cys His, Asn and Gln, tBu for Tyr Thr, Asp, Glu and Ser; Boc for Lys and the indole N of Trp, Pmc for Arg. Activation of the carboxyl groups was achieved using, TBTU/HOBt/DIPEA, all couplings were carried out in DMF. Deprotection of the Fmoc groups was achieved with 20% Piperidine in DMF. Cleavage of the peptides from the resin was carried out with 5% Anisole/5% Thioanisole/5% EDT/3% Water/2% TES in TFA for 1 hour. The peptides were purified by RP-HPLC using a 40 mm×210 mm Deltapak C18 radial compression column on a Waters Deltaprep 4000 and characterised by MALDI-TOF on a Kratos Maldi 3 and by AAA.

For dendrimers Fmoc Lys(Fmoc)-OH is attached by the methods above and gives both and amino groups free for peptide elongation. Quantities of Fmoc amino acids used have to be increased accordingly.

Rink Amide Linker=p-[(R,S-[1-(9H-Fluoren-9-yl)-methoxyformamido]-2,4-dimethoxybenzyl]-phenoxyacetic acid

Fmoc=9-Fluorenylmethoxycarbonyl

Trt=Trityl, Triphenylmethyl

tBu=tertiary butyl

Boc=tertiary butyloxycarbonyl

Pmc=2,2,5,7,8-Pentamethylchroman-6-sulphonyl

TBTU=2-(1H-Benzotriazole-1-yl)-1,1,3,3-tetramethyluronium tetrafluoroborate

HOBt=N-Hydroxybenzotriazole

DIPEA=Diisopropylethylamine

DMF=N,N Dimethylformamide

EDT=Ethanedithiol

TES=Triethylsilane

TFA=Trifluoroacetic acid

RP-HPLC=Reverse phase high performance liquid chromatography

MALDI-TOF=Matrix assisted laser desorption ionisation—time of flight

AAA=Amino acid analysis

Fmoc-Lys(Fmoc)-OH=di-9-fluorenylmethoxycarbonyl lysine

The following peptides were synthesized in this manner:

(1) N-Acetyl-VEGF 6-Gly-Cys
Ac-TMQIMRIKPHQGQHIGEMSGC-NH₂
(2) N-Acetyl-Cys-Gly-VEGF 7
Ac-CGTMQIMRIKPHQGQ-NH₂
(3) N-Acetyl-VEGF 8-Gly-Cys
Ac-TMQIMRIKPHQGQGC-NH₂
(4) N-Acetyl-Cys-Gly-VEGF 9
Ac-CGMRIKPHQGQHIGEMS-NH2

### EXAMPLE 2

### Conjugation Procedure

To Keyhole Limpet Haemocyanin (KLH) solution in phosphate buffered saline (PBS), a 150 molar excess of S-MBS, m-Maleimidobenzoyl-N-hydroxysulphosuccinimide ester is added and stirred for 0.5 hours at 20-25° C. in a sealed vial.

Excess S-MBS cross-linker is removed by chromatography (gel exclusion using G-25 Sephadex course matrix) in PBS. The activated KLH peak is collected, assayed for free maleimido groups and used as below.

To the resulting KLH carrier protein solution a 2 molar excess of VEGF derivative peptide is added. The resulting solution is stirred for up to 2 hours at 2-8° C. in a sealed container.

The conjugate is purified from free peptide by gel exclusion chromatography as above.

The final conjugate is diluted to a working concentration and formulated as desired.

Following this procedure, VEGF derivatives (1) to (4) of Example 1 were conjugated individually to aliquots of activated KLH.

### EXAMPLE 3

### Immunisation Studies

The four VEGF derivatives of Example 1, conjugated individually to aliquots of KLH as described in Example 2, were formulated by adsorption to 0.4% (w/v) aluminium hydroxide gel (Alhydrogel, Superfos s/a, Denmark) in a normal saline (0.9% (w/v)) vehicle. All conjugates were used as 25 μg/ml peptide equivalent solution. C57BL/6 Mice were used in 5 treatment groups, with 23 mice per group. The treatment groups received:

Group A: Alhydrogel/Saline placebo vaccine 0.2 ml/mouse.

Group B: N-Acetyl-VEGF 6-Gly-Cys at 5 μg peptide equivalent/derivative immunotherapeutic per mouse in 0.2 ml vaccine.

Group C: N-Acetyl-Cys-Gly-VEGF 7 at 5 μg peptide equivalent/derivative immunotherapeutic per mouse in 0.2 ml vaccine.

Group D: N-Acetyl-VEGF 8-Gly-Cys at 5 μg peptide equivalent/derivative immunotherapeutic per mouse in 0.2 ml vaccine.

Group E: N-Acetyl-Cys-Gly-VEGF 9 at 5 μg peptide equivalent/derivative immunotherapeutic per mouse in 0.2 ml vaccine.

The route of used was subcutaneous and each mouse received 3 separate doses of the specified test article during the course of the study. The bodyweight of each mouse is recorded on each dosing day and at termination the study.

Experimental Procedure

On day −50 and subsequently on days −28 and −7, the mice received a single subcutaneous dose of the vehicle, or the test articles. Sera was prepared for analysis on days −1 and +14 of the study.

Each sample of blood was collected by cardiac puncture into serum tubes, allowed to clot, then centrifuged to yield serum within 45 minutes of sampling.

Serum samples were frozen at approximately −20° C. as soon as possible.

TABLE 1
Time schedule of study procedures.

| | | Sera |
|---|---|---|
| Day | Treatment | Sample |
| −50 | Test articles, vehicle | |
| −28 | Test articles, vehicle | |
| −7 | Test articles, vehicle | |
| −1 | No dosing | + |
| 14 | No dosing | + |

Each serum sample was assayed for generation by treatment, of an antibody response by titration of anti-VEGF peptide-antibodies present in the sera by Enzyme Linked ImmunoSorbant Assay (ELISA).

This assay was performed as follows:

Coat the 96 well Nunc Maxisorp microtitre plates with 100 μl detection substrate e.g. N-Acetyl-VEGF 6-Gly-Cys-BSA (10 μg peptide equivalent/well) diluted in PBS, for 1 hour at 20-25° C. At the same time place 100 μl PBS into separate wells to act as a substrate blank.

Wash the plates 3 times with 200 μl Phosphate Buffered Saline PBS)/0.1% Tween 20.

Dilute the serum samples to a suitable dilution with PBS/0.1% Tween 20. Typical dilutions would be as follows:

i) 1/100-5 μl mouse sera +495 μl PBS/0.1% Tween 20

ii) 1/1000-20 μl (i)+180 μl PBS/0.1% Tween 20

iii) 1/2000-10 μl (i)+190 μl PBS/0.1% Tween 20

iv) 1/5000-4 μl (i)+196 μl PBS/0.1% Tween 20

Load the appropriate diluted sera (100 μl) to appropriate wells and incubate at for 1 hour at 20-25° C. to permit substrate:antibody binding.

Wash the plates 3 times with 200 μl PBS/0.1% Tween 20.

Dilute rabbit anti-mouse IgG peroxidase conjugate 1:5000 in PBS/0.1% Tween 20 i.e. 1 μl IgG peroxidase +5 mls PBS/0.1% Tween 20. This binds to the mouse serum antibody and allows antibody detection.

Add 100 μl of the diluted IgG peroxidase to the appropriate wells and leave for 45 minutes at 20-25° C.

Wash the plates 3 times with 200 μl PBS.

250 μl aliquot of the perodidase substrate 3,3¹,5,5¹,-tetra methyl benzidine (TMB) to 25 mls 0.1M sodium acetate buffer pH5.5 with 4 μl 30% hydrogen peroxide.

Add 100 μl of the prepared TMB substrate to the appropriate wells, including the blank wells. A colour producing reaction occurs where antibody/substrate binding has occurred. Leave for 15 minutes at 20-25° C., then terminate the reaction with 50 μl 10% sulphuric acid added to each well.

The plate was read for absorbency of light at 405 nm generated by the reaction of the peroxidase enzyme on the TMB substrate and is proportional to the amount of primary (anti-VEGF) antibody bond. The resultant absorbency readings were analysed by a statistical package (SAS Institute 1997) to determine titre.

TABLE 2
Antibody titres (+/− sem) for each sera sample day.

| | Mean Antibody titre (by ELISA) | |
|---|---|---|
| Treatment Group | Day −1 | Day 14 |
| A | 0 | 0 |
| B | 17567 ± 2082 | 19870 ± 2413 |
| C | 7952 ± 3542 | 17355 ± 1998 |
| D | 11315 ± 3897 | 18144 ± 2163 |
| E | 15781 ± 4331 | 32625 ± 2144 |

For Day −1 data n = 3, for Day 14 data n = 20.

In parallel with the antibody titre data, all animals were examined for gross physiological changes in body weight, laboured breathing and general appearance, as an overall assessment of toxic or harmful effects.

No adverse effects were recorded for any of the treatment groups, showing that the VEGF treatments are effective in generating anti-VEGF antibodies, without harmful physiological effects in the animals.

### EXAMPLE 4

### Murine Anti-Metastatic Studies

To evaluate the anti-metastatic potential of the VEGF immunotherapeutic vaccines, the vaccinated mice described in Example 3 were treated as described here. In addition an additional group of mice having received neither vaccine nor placebo were treated in parallel as the murine model control group.

Experimental Procedure

Each mouse was injected intravenously with 0.1 ml suspension of B16/F10 melanoma cells on day 0 of the study. On day 14 of the study the mice were sacrificed and the lungs removed from each mouse. The lungs were weighed prior to fixing in Bouin's solution to visualise any resulting melanoma colonies from administration of the B16/F10 cells. Following fixation the number of colonies present on the surface of the lungs prepared from each mouse, was counted.

TABLE 3
Effect of each VEGF vaccine treatment on murine lung weight
following administration of B16/F10 melanoma cells.

| Treatment Group | Lung weight (mg) | % Change * |
|---|---|---|
| Murine Control | 217.1 ± 4.56 | −4.5 |
| A | 227.2 ± 9.47 | 0 |
| B | 237.7 ± 7.46 | +4.6 |
| C | 250.3 ± 6.76 | +10.2 |
| D | 232.3 ± 6.21 | +2.2 |
| E | 250.6 ± 9.03 | +10.3 |

Data are expressed as mean ± sem (n = 19 − 20).
* As compared to Treatment Group A.

TABLE 4
Effect of each VEGF vaccine on the murine model lung colony
count following administration of B16/F10 melanoma cells.

| Treatment Group | Colony count | % Change |
|---|---|---|
| A | 81.2 ± 3.79 | — |
| B | 59.45 ± 6.28 | 26.8 |
| C | 74.3 ± 6.42 | 8.5 |
| D | 58.69 ± 5.36 * | 27.5 |
| E | 53.2 ± 8.46 ** | 34.5 |

Data are expressed as mean ± sem (n = 19 − 20).
* P < 0.05, ** P < 0.01, compared to Treatment Group A using Kruskal-Wallis and Dunn's test.

Table 2 demonstrates a clear generation of the respective anti-VEGF6, VEGF7, VEGF8 and VEGF9 IgG molecules by the titre results reported. This shows that the vaccines investigated caused active immunisation to the target peptides, confirmed by comparison with control treatment group A, immunised with placebo only yielding a anti-VEGF peptide titre of 0.

Table 3 demonstrates that lung weight increased for all groups immunised with the VEGF conjugates. Compared to the murine model control group, vaccine treated groups B, C and D demonstrated an increased in lung weight of upto 15%. Whilst the immunised placebo control group showed a marginal increase in lung weight compared to the murine model control receiving neither vaccine nor placebo.

Table 4 demonstrates that the lung melanoma colony count for groups immunised with the VEGF conjugates, decreased by over a third compared to the placebo group treated with vaccine placebo alone (Group A). The greatest reduction in colony count, indicating the best control of metastasis and was also statistically significant, was for the groups treated with vaccines comprising VEGF8 and VEGF9.

All VEGF vaccine treated groups had good anti-VEGF peptide IgG titres, at Day 14 of the study. Nonetheless, IgG generated following immunisation with formulations VEGF8 and VEGF9 demonstrated a distinctly inherent capacity to significantly control tumour spread. This emphasizes that the IgG molecules raised to peptides VEGF8 and VEGF9 have potentially the most potent activity for controlling tumour metastasis. These data demonstrate specific interaction of IgG molecules raised to peptides VEGF8 and VEGF9 with native VEGF in the murine model used for these studies.

1. An immunogenic conjugate comprising at least one vascular endothelial growth factor (VEGF) peptide moiety coupled to a carrier.

2. A conjugate according to claim 2 for use in therapy or prophylaxis.

3. A conjugate as claimed in claim 1 for combatting tumours and tumour metastasis.

4. A conjugate as claimed in claim 1 wherein said VEGF peptide moiety comprises an amino acid sequence having at least 80% homology with the whole or a section of a native VEGF sequence.

5. A conjugate as claimed in claim 4 wherein said section of a native VEGF sequence is a section of 8 to 100 amino acids.

6. A conjugate as claimed in claim 4 wherein said section of a native VEGF sequence is a section of 12 to 25 amino acids.

7. A conjugate as claimed in claim 4 wherein said degree of homology is with a section of a native VEGF sequence overlapping, abutting or adjacent a glycosylation site.

8. A conjugate as claimed in claim 7 wherein said section includes at least 12 of the first 16 amino acid residues from said glycosylation site in the N-terminal direction.

9. A conjugate as claimed in claim 1 wherein the VEGF peptide moiety comprises an oligopeptide comprising at least part of SEQ ID No. 1: TEESNITMQI MRIKPHQGQH IGEMSFLQHN.

10. A conjugate as claimed in claim 1 wherein the VEGF peptide moiety comprises an oligopeptide of formula:
(T)ₐ(M)_{b}(O)_{c}(I)_{d}MRIKPHQGQ (H)ₑ(I)_{f}(G)_{g}(E)ₕ(M)ᵢ(S)ⱼ(F)ₖ(L)ₗ(O)ₘ
where:
a to m are each 0 or 1
but a to c and f to m may not be 1 unless the sequence so created corresponds to a sequence in SEQ ID No. 1
e to g are 1

11. A conjugate as claimed in claim 10 wherein e to j are 1.

12. A conjugate as claimed in claim 1 wherein said VEGF peptide moiety is coupled via its N-terminal end to the carrier.

13. A conjugate as claimed in claim 1 where said carrier is selected from the group consisting of the purified protein derivative of tuberculin, tetanus toxoid, diptheria toxoid, keyhole limpet haemocyanin, glutathione S-transferase and derivatives thereof.

14. The use of an immunogenic conjugate as claimed in claim 1 comprising at least one vascular endothelial growth factor peptide moiety coupled to a carrier for the manufacture of a medicament for use in combatting tumours.

15. A vascular endothelial growth factor derivative comprising at least one VEGF peptide moiety coupled to a peptide carrier-binding moiety.

16. A nucleic acid molecule coding for a vascular endothelial growth factor derivative comprising at least one VEGF peptide moiety coupled to a peptide carrier-binding moiety according to claim 15, and nucleic acid molecules with sequences complementary thereto.

17. An expression vector comprising a nucleic acid molecule according to claim 16.

18. A pharmaceutical composition comprising a conjugate according to claim 1 together with one or more pharmaceutically acceptable carriers or excipients.

19. A method of combatting tumours in a human or non-human subject comprising administering to said subject an effective amount of a conjugate as defined in claim 1.

20. A method for achieving maximal blockade of VEGF in a human or non-human subject comparable to or exceeding that achieved by chemo- or radiotherapy, said method comprising administering to said subject an effective amount of a VEGF conjugate as claimed in claim 1.
